(19) United States
(12) Patent Application Publication
(10) Pub. No.: US 2023/0414684 A1
(43) Pub. Date: Dec. 28, 2023

(54) COMPOSITION FOR PREVENTING OR TREATING COLORECTAL CANCER COMPRISING MIXED STRAIN OF PROBIOTICS

(71) Applicant: MEDIOGEN CO., LTD., Jecheon-si (KR)

(72) Inventors: Nam Soo Paek, Jecheon-si (KR); Chang Ho Kang, Jecheon-si (KR)

(21) Appl. No.: 18/311,691

(22) Filed: May 3, 2023

(30) Foreign Application Priority Data
May 4, 2022 (KR) 10-2022-0055490

(51) Int. Cl. A61K 35/747; A61K 35/745; A61P 35/00

(52) U.S. Cl. CPC A61K 35/747; A61K 35/745; A61P 35/00

(57) ABSTRACT

Provided is a composition for preventing or treating colorectal cancer including a mixed strain of probiotics as an active ingredient, specifically, a composition for preventing, alleviating or treating colorectal cancer including a mixed killed strain of L. casei, L. reuteri or Bi. bifidum. According to the present disclosure, the composition is confirmed to have excellent effects of activating apoptosis-related signaling molecules in colorectal cancer cells and inhibiting the growth of tumors derived from colorectal cancer cells, and thus may be usefully used in various industrial fields such as medicines and foods, for prevention, alleviation, or treatment of colorectal cancer.

### CROSS-REFERENCE TO RELATED APPLICATIONS

This application is based on and claims priority from Korean Patent Application No. 10-2022-0055490, filed on May 4, 2022, with the Korean Intellectual Property Office, the disclosure of which is incorporated herein in its entirety by reference.

### FIELD OF THE INVENTION

The present disclosure relates to a composition for preventing or treating colorectal cancer including a mixed strain of probiotics as an active ingredient, and more particularly, to a composition for preventing, alleviating, or treating colorectal cancer including a mixed killed strain of L. casei, L. reuteri or Bi. bifidum.

### BACKGROUND OF THE INVENTION

Para-biotics are killed lactic acid bacteria that do not have survival activities, and are also called names such as killed lactic acid bacteria, tyndallized lactic acid bacteria, and heat-treated lactic acid bacteria. The para-biotics do not survive in the body, but contains microbial cell components as they are, which may help for health, such as enzymes, glycoproteins, short-chain fatty acids, and DNA components. Accordingly, the para-biotics have recently attracted attention because they may provide various physiological advantages to humans through these bioactive compounds.

First of all, in most cases, adverse reactions such as an increase in blood lactic acid concentration or gas induction that may occur after ingestion of lactic acid bacteria are caused by the survival activity of probiotic bacteria, so that the para-biotics are free from these adverse reactions because lactic acid bacteria are consumed in a dead state. Accordingly, the para-biotics have high stability compared to probiotics represented by probiotic products, and thus have the greatest advantage of being able to consume hundreds of billions to trillions of high concentrations. In addition, the para-biotics have excellent heat resistance and high stability to the external environment, so that there are remarkable advantages that it is easier to store than existing probiotic products and the expiration date may be extended.

Based on these advantages, the para-biotics are showing rapid growth in fields such as health functional foods, food additives, and pharmaceuticals to which existing lactic acid probiotics have been applied. In addition, since regulations on the use of antibiotics in the pharmaceutical field are being strengthened, there is usefulness as an alternative, and since there are still only a handful of companies that have entered the production of para-biotics products in earnest, the marketability and growth potential may be great.

Despite the fact that these killed lactic acid bacteria are more advantageous in terms of industrial availability, studies on the mechanisms of action of these physiological effects in cells and the human body or on more diverse physiological functions have been currently not being conducted much not only in Korea but also worldwide.

### SUMMARY OF THE INVENTION

An objective to be achieved by the present disclosure is to provide a method for alleviating or treating colorectal cancer, comprising administering a pharmaceutical composition, a health functional food composition or a feed additive composition including a killed strain of L. casei MG4584 (accession number: KCTC14423BP) and a killed strain of L. reuteri MG5346 (accession number: KCTC14507BP), and a method for preparing the composition.

Another objective to be achieved by the present disclosure is to provide a method for alleviating or treating colorectal cancer, comprising administering a pharmaceutical composition, a health functional food composition or a feed additive composition including a killed strain of L. casei MG4584 (accession number: KCTC14423BP), a killed strain of L. reuteri MG5346 (accession number: KCTC14507BP), and a killed strain of Bi. bifidum MG731 (accession number: KCTC13452BP), and a method for preparing the composition.

An exemplary embodiment of the present disclosure provides a pharmaceutical composition for preventing or treating colorectal cancer including a killed strain of L. casei MG4584 (accession number: KCTC14423BP) and a killed strain of L. reuteri MG5346 (accession number: KCTC14507BP).

The composition may further include a killed strain of Bi. bifidum MG731 (accession number: KCTC13452BP).

The killed strain may be heat-killed.

The heat-killing may be heat treatment at a temperature of 70° C. to 130° C. for 10 minutes to 60 minutes.

The L. casei MG4584 killed strain and the L. reuteri MG5346 killed strain may be included in a cell number ratio of 1:1 to 2.

The L. casei MG4584 killed strain, the L. reuteri MG5346 killed strain, and the Bi. bifidum MG731 killed strain may be included in a cell number ratio of 1:1 to 2:1 to 2.

The composition may have tumor growth inhibitory activity or apoptosis inducing activity of cancer cells.

Another exemplary embodiment of the present disclosure provides a health functional food composition for preventing or alleviating colorectal cancer including a killed strain of L. casei MG4584 (accession number: KCTC14423BP) and a killed strain of L. reuteri MG5346 (accession number: KCTC14507BP).

The composition may further include a killed strain of Bi. bifidum MG731 (accession number: KCTC13452BP).

Yet another exemplary embodiment of the present disclosure provides a feed additive composition for preventing or alleviating colorectal cancer including a killed strain of L. casei MG4584 (accession number: KCTC14423BP) and a killed strain of L. reuteri MG5346 (accession number: KCTC14507BP).

The composition may further include a killed strain of Bi. bifidum MG731 (accession number: KCTC13452BP).

Still another exemplary embodiment of the present disclosure provides a method for preparing a composition for preventing, alleviating or treating colorectal cancer including mixing heat-killed strains, in which the strains are L. casei MG4584 (accession number: KCTC14423BP) and L. reuteri MG5346 (accession number: KCTC14507BP).

The strain may further include Bi. bifidum MG731 (accession number: KCTC13452BP).

The preparing method may further include lyophilizing and powdering the heat-killed strains.

The heat-killed strains may be included in a cell number ratio of 1:1 to 2:1 to 2.

According to the exemplary embodiments of the present disclosure, the composition is confirmed to have excellent effects of activating apoptosis-related signaling molecules in colorectal cancer cells and inhibiting the growth of tumors derived from colorectal cancer cells, and thus can be usefully used in various industrial fields such as medicines and foods, for prevention, alleviation, or treatment of colorectal cancer.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIGS. 1A-1B are diagrams illustrating an apoptosis effect of killed strains of Bifidobacterium and Lactobacillus on human colorectal cancer RKO cells (Mean±SD, n=3, *: p<0.05). FIG. 1A: Percentage distribution by Annexin V-FITC and PI staining reaction, FIG. 1B: Comparison of Total apoptosis in percentage.

FIGS. 2A-2D are diagrams illustrating a growth inhibitory effect on RKO cell-derived tumors in a mixed group of two killed strains and a mixed group of three killed strains (Mean±SD, n=5, *: p<0.05). FIG. 2A: Tumor images of xenograft model animals, FIG. 2B: 3-week tumor growth curve, FIG. 2C: Comparison of tumor weights, FIG. 2D: 3-week body weight change.

FIGS. 3A-3E are diagrams illustrating a comparison of apoptosis signal molecule activities of a mixed group of two killed strains and a mixed group of three killed strains in RKO cell-derived tumor tissues by Western blotting (Mean±SD, n=3, *: p<0.05). FIG. 3A: Western blot data, FIG. 3B: Comparison of cleaved caspse-9/caspase-9 expression, FIG. 3C: Comparison of cleaved caspse-7/caspase-7 expression, FIG. 3D: Comparison of cleaved caspse-3/caspase-3 expression; FIG. 3E: Comparison of cleaved PARP/PARP expression.

FIG. 4 is a diagram illustrating a comparison of apoptosis signal molecule activities according to the administration of a single killed strain or a mixed killed strain in RKO cell-derived tumor tissues as immunohistochemical (IHC) staining images.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Unless otherwise contrarily defined, all terms used herein including technological or scientific terms have the same meanings as those generally understood by a person with ordinary skill in the art.

Terms which are defined in a generally used dictionary should be interpreted to have the same meaning as the meaning in the context of the related art, and are not interpreted as an ideal meaning or excessively formal meanings unless otherwise clearly defined in the present application. Further, in describing the present disclosure, a detailed explanation of known related technologies may be omitted to avoid unnecessarily obscuring the subject matter of the present disclosure.

The present inventors identified a molecular mechanism of the antitumor activity of a heat-killed Bifidobacterium or Lactobacillus strain on colorectal cancer, confirmed a synergistic effect by the combination of the killed strains, and invented a mixed killed strain that exhibited therapeutic efficacy for colorectal cancer.

Specifically, the present disclosure relates to a pharmaceutical composition for preventing or treating colorectal cancer including a killed strain of L. casei MG4584 (accession number: KCTC14423BP) and a killed strain of L. reuteri MG5346 (accession number: KCTC14507BP).

The composition may further include a killed strain of Bi. bifidum MG731 (accession number: KCTC13452BP).

In the present disclosure, the “colorectal cancer” means a tumor that occurs anywhere in the colon or rectal mucosa, and specifically, tumors that occur in the rectum, sigmoid colon, descending colon, transverse colon, ascending colon, appendix, anal canal, and the like are collectively referred to as colorectal cancer, depending on a location of occurrence, and colon cancer, rectal cancer, and colorectal cancer may be used interchangeably.

According to one embodiment of the present disclosure, in an animal model in which colorectal cancer has occurred through subcutaneous injection of colorectal cancer cells or colorectal cancer cell lines, apoptosis-inducing activity or tumor growth inhibitory activity of the Lactobacillus killed strain or Bifidobacterium killed strain was confirmed. It was confirmed that when the mixed killed strain was treated rather than the killed strain alone, the synergistic anticancer activity was exhibited and the excellent therapeutic effect of the killed strain combination against colorectal cancer was demonstrated.

In the present disclosure, the “killed strain” is a form in which the growth of bacteria is prevented from occurring by methods such as heat treatment, chemical treatment, and radiation treatment after culturing live strains under certain conditions, and is dead cells of bacteria and lysates of lactic acid bacteria at the same time. That is, the killed strain includes the cytoplasms, cell walls, bacteriocins, polysaccharides, organic acids, etc. including metabolites, in a killed state. Most of the physiologically active ingredients of lactic acid bacteria are concentrated in the cell walls of lactic acid bacteria, and these cell walls are taken up by intestinal M cells after the lactic acid bacteria are killed, so that killed strains are called parabiotics in the sense that the killed strains play a role similar to live strains. In the present disclosure, the killed strains are meant to include all of killed strains of lactic acid bacteria, killed strain cultures, killed strain extracts, and killed strain cell ingredients.

In the present disclosure, the “mixed killed strain” means a mixture of killed strains, and may be a mixture in which killed strains of each lactic acid bacteria are mixed or a mixture in which each lactic acid bacteria is mixed and then killed, and is not particularly limited. The mixed killed strain of the present disclosure may be a mixture of a killed strain of L. casei MG4584 and a killed strain of L. reuteri MG5346 or a mixture of a killed strain of L. casei MG4584, a killed strain of L. reuteri MG5346, and a killed strain of Bi. bifidum MG731.

The L. casei MG4584 was patent-deposited at the Korean Collection for Type Cultures (KCTC), 181 Ipsin-gil, Jeongeup-si, Jeollabuk-do, Korea, and received an accession number KCTC14423BP.

The present inventors deposited L. casei MG4584 with the Korean Collection for Type Cultures (KCTC) (address: 181 Ipsin-gil, Jeongeup-si, Jeollabuk-do, Korea), an international depositary authority, on Jan. 5, 2021 under accession number KCTC14423BP.

The L. reuteri MG5346 was patent-deposited at the Korean Collection for Type Cultures (KCTC), 181 Ipsin-gil, Jeongeup-si, Jeollabuk-do, Korea, and received an accession number KCTC14507BP.

The present inventors deposited L. reuteri MG5346 with the Korean Collection for Type Cultures (KCTC) (address: 181 Ipsin-gil, Jeongeup-si, Jeollabuk-do, Korea), an international depositary authority, on Mar. 22, 2021 under accession number KCTC14507BP.

The Bi. bifidum MG731 was patent-deposited at the Korean Collection for Type Cultures (KCTC), 181 Ipsin-gil, Jeongeup-si, Jeollabuk-do, Korea, and received an accession number KCTC13452BP.

The present inventors deposited Bi. bifidum MG731 with the Korean Collection for Type Cultures (KCTC) (address: 181 Ipsin-gil, Jeongeup-si, Jeollabuk-do, Korea), an international depositary authority, on Jan. 4, 2018 under accession number KCTC13452BP.

In the present disclosure, the killed strains may be obtained by killing the lactic acid bacteria by heat treatment, radiation treatment, chemical treatment, etc., and preferably heat-killed at a temperature of 70° C. to 130° C. for 10 minutes to 60 minutes.

The mixed killed strain of the present disclosure may include a killed strain of L. casei MG4584 and a killed strain of L. reuteri MG5346 in a cell number ratio of 1:1 to 2. Alternatively, the mixed killed strain of the present disclosure may include a killed strain of L. casei MG4584, a killed strain of L. reuteri MG5346, and a killed strain of Bi. bifidum MG731 in a cell number ratio of 1:1 to 2:1 to 2.

It is preferred to mix the killed strains in the cell number ratio at a concentration of 1×10⁸ to 1×10¹⁰ cells/ml, respectively, or to use the composition including the mixed killed strain so that the concentration of killed lactic acid bacteria is adjusted to 1×10⁸ to 1×10¹⁰ cells/ml using an appropriate diluent. The mixed killed strain may exhibit the optimum anticancer activity at the concentration 1×10⁸ to 1×10¹⁰ cells/ml and secure a homogeneous appearance.

The composition of the present disclosure may have tumor growth inhibitory activity or apoptosis inducing activity of cancer cells.

According to one embodiment of the present disclosure, when the colorectal cancer cells are treated with the L. casei MG4584 killed strain, the L. reuteri MG5346 killed strain and the Bi. bifidum MG731 killed strain, it is confirmed that the apoptosis inducing activity is increased. When the killed strain is administered alone at 1×10⁹ cells/mouse or a mixture of killed strains mixed at 1×10⁹ cells/mouse is administered to an animal model in which colorectal cancer is developed, it was confirmed that when the mixed killed strains were administered rather than the killed strain alone, improved synergy in tumor growth inhibitory activity and apoptosis inducing activity was exhibited.

In addition, since the mixed killed strain is involved in a Caspase Cascade pathway, which is an apoptosis-related signal, to increase cleaved caspse/caspase, it is confirmed that the tumor growth inhibition of the animal model is active by the induction of apoptosis of the mixed killed strain other than the necrosis of the tumor. Therefore, it is possible to provide a pharmaceutical composition for preventing or treating colorectal cancer including the mixed killed strain according to the present disclosure.

The pharmaceutical composition of the present disclosure may be administered to a patient diagnosed with colorectal cancer or confirmed to have a risk of developing colorectal cancer. The prevention or treatment of colorectal cancer using the composition may further have the activity of reducing metastasis of cancer cells in addition to the activity of delaying and/or inhibiting tumor formation or tumor growth of cancer cells.

The composition may be performed alone or in combination with other therapies, for example, surgery, radiation therapy, gene therapy, immunotherapy (e.g., targeted antibody immunotherapy, CAR-T cell therapy), oncolytic viruses, bone marrow transplantation, stem cell transplantation, hormone therapy, targeted therapy, cryotherapy, ultrasound therapy, photodynamic therapy, and chemotherapy.

Additionally, persons having a high risk of developing proliferative diseases may receive treatment to inhibit and/or delay the occurrence of the diseases. The anticancer activity of the composition according to the present disclosure may be effective when combined with more direct anticancer agents. Accordingly, in a specific embodiment, the present disclosure may provide a composition including a mixed killed strain and an anticancer agent.

The mixed killed strain of L. casei MG4584 and L. reuteri MG5346 or the mixed killed strain of L. casei MG4584, L. reuteri MG5346, and Bi. bifidum MG731 as the active ingredient of the composition according to the present disclosure may be included in an amount of 0.00001 wt % to 100 wt %, 0.001 wt % to 99.9 wt %, or 0.1 wt % to 99 wt %, and more preferably 1 wt % to 50 wt % with respect of the entire anticancer composition.

In the present disclosure, the pharmaceutical composition may be administered to mammals including humans through various routes. The administration method may be any method commonly used, and may be administered by, for example, oral, dermal, intravenous, intramuscular or subcutaneous routes, and preferably orally.

The composition may include a pharmaceutically acceptable excipient or carrier. Acceptable carriers or diluents for therapeutic use are well-known in the pharmaceutical field. Examples of suitable carriers include lactose, starch, glucose, methyl cellulose, magnesium stearate, mannitol, and sorbitol. Examples of suitable diluents include ethanol, glycerol and water. The pharmaceutical carrier, excipient or diluent may be selected with reference to an intended route of administration and standard pharmaceutical practice. The pharmaceutical composition may include any suitable binder, lubricant, suspending agent, coating agent, and solubilizing agent, as or in addition to the carrier, excipient or diluent. The carrier, the excipient, and the diluent may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, etc.

In the case of preparing or formulating the pharmaceutical composition, the formulations may be prepared by using diluents or excipients, such as a filler, an extender, a binder, a wetting agent, a disintegrating agent, and a surfactant, which are generally used. Solid formulations for oral administration include tablets, pills, powders, granules, capsules, etc., and these solid formulations may be prepared by mixing at least one excipient, for example, starch, calcium carbonate, sucrose, lactose, and gelatin with the composition. Further, lubricants such as magnesium stearate and talc are used in addition to simple excipients.

Liquid formulations for oral administration may correspond to suspensions, solutions, emulsions, syrups, and the like, and may include various excipients, for example, a wetting agent, a sweetener, an aromatic agent, and a preservative, in addition to water and liquid paraffin which are commonly used as simple diluents. Formulations for parenteral administration include sterilized aqueous solutions, non-aqueous solvents, suspensions, emulsions, lyophilized agents, suppositories, and the like. As the non-aqueous solution and the suspension, propylene glycol, polyethylene glycol, vegetable oil such as olive oil, and injectable ester such as ethyl oleate may be used. As a base of the suppository, witepsol, macrogol, tween 61, cacao butter, laurinum, glycerogelatin, and the like may be used. The ingredients may be added in combination with active ingredients, that is, a mixed killed strain, cultures thereof, extracts thereof, or strain ingredients thereof.

In addition, the composition may be formulated and used in the form of oral formulations, such as powders, granules, tablets, capsules, suspensions, emulsions, syrups, and aerosols, external preparations, suppositories, and sterile injectable solutions according to a general method.

A suitable dose of the pharmaceutical composition according to the present disclosure may be variously prescribed by factors, such as a formulation method, an administration method, age, body weight, and sex of a patient, a morbid condition, food, an administration time, an administration route, an excretion rate and response sensitivity, and may be administered once or several times a day at regular time intervals depending on the judgment of a doctor or pharmacist. For example, the daily dose may be 0.1 to 10,000 mg/kg, and preferably 1 to 2,000 mg/kg, based on the active ingredient content. The dose is an example of an average case, and an optimal dosage to be administered may be determined by those skilled in the art, and may be adjusted by experts in the art according to various factors including the type of disease, the severity of disease, the content of active ingredients and other ingredients contained in the composition, a type of formulation, age, body weight, general health condition, sex and diet of a patient, an administration time, a route of administration, a secretion rate of a composition, duration of treatment, and drugs used concurrently.

In addition, it is possible to provide a health functional food composition for preventing or alleviating colorectal cancer including the mixed killed strain according to the present disclosure.

The health functional food composition includes all forms, such as functional foods, nutritional supplements, health functional foods, and food additives. The type of food composition may be prepared in various forms according to general methods known in the art.

The functional foods may be prepared in the form of beverages, fruits and their processed foods, fish, meat and their processed foods, breads and noodles, fruit juices, various drinks, cookies, taffy, dairy products, edible vegetable oils, margarine, vegetable proteins, retort foods, frozen foods, various seasonings, etc.

The health functional food is the same term as food for special health use (FoSHU), and refers to food with a high medical/care effect, which is processed so that a bioregulatory function is effectively shown in addition to nutrition supply. Here, the ‘function(ality)’ refers to regulating nutrients with respect to the structure and function of the human body or obtaining effects useful for health applications such as physiological action. The health food refers to food that has a more active health maintenance or promotion effect than general food, and the health supplement food refers to food for the purpose of health supplement. In some cases, the terms of the health functional food, the health food, and the health supplement food may be used interchangeably.

Specifically, the health functional food is food prepared in encapsulation, powder, suspension, etc., and means bringing a specific effect on health when ingested, but has an advantage of having no side effects that may occur when taking drugs for a long time by using the food as a raw material, unlike general drugs.

Preferred content of the food composition of the present disclosure is not limited thereto, but may be, for example, 0.01 to 80 wt % of the finally prepared food, and preferably 0.01 to 50 wt % of the finally prepared food.

The food composition of the present disclosure may be prepared by methods which are commonly used in the art and may be prepared by adding raw materials and ingredients which are commonly added in the art. In addition, the food composition may be prepared without limitation in various types of formulations as long as the formulations are recognized as food. In addition, the food composition may further include a physiologically acceptable carrier, and the type of carrier is not particularly limited, and any carrier commonly used in the art may be used. In addition, the food composition may include an additional ingredient to be commonly used in the food composition to enhance smell, taste, sight, and the like. For example, the food composition may include vitamins A, C, D, E, B1, B2, B6, and B12, niacin, biotin, folate, panthotenic acid, and the like. In addition, the food composition may include minerals such as zinc (Zn), iron (Fe), calcium (Ca), chromium (Cr), magnesium (Mg), manganese (Mn), and copper (Cu); and amino acids such as lysine, tryptophan, cysteine, and valine. In addition, the food composition may include food additives, such as preservatives (potassium sorbate, sodium benzoate, salicylic acid, sodium dehydroacetate, etc.), disinfectants (bleaching powder and highly bleaching powder, sodium hypochlorite, etc.), antioxidants (butylhydroxyanisole (BHA), butylhydroxytoleuene (BHT), etc.), colorants (tar color, etc.), coloring agents (sodium nitrite, sodium nitrite, etc.), bleach (sodium sulfite), seasoning (MSG sodium glutamate, etc.), sweeteners (dulcin, cyclamate, saccharin, sodium, etc.), flavorings (vanillin, lactones, etc.), expanding agents (alum, potassium D-bitartrate, etc.), reinforcing agents, emulsifying agents, thickening agents, coating agents, gum base agents, foam inhibitors, solvents, and improving agents. The additives may be selected according to a type of food and used in an appropriate amount.

In addition, it is possible to provide a feed additive composition for preventing or alleviating colorectal cancer including the mixed killed strain according to the present disclosure.

The feed added with the composition may be provided to companion animals or livestock for the purpose of preventing cancer, diagnosed with colorectal cancer, or confirmed to be at risk of developing colorectal cancer. The mixed killed strain, as a raw material of the composition, may be used in combination with conventionally known feed additives or feeds, and may also include various excipients added to known feed additives or feeds. The composition may further include nutrients, antiseptics, preservatives, antibiotics, trace nutrient ingredients such as vitamins, which are commonly used in animal feed. The nutrients, antiseptics, preservatives, antibiotics, trace nutrient ingredients, etc. may be arbitrarily selected and used by those skilled in the art in an amount commonly added to feed.

Still another aspect of the present disclosure provides a method for preparing a composition for preventing, alleviating or treating colorectal cancer including mixing heat-killed strains, in which the strains are L. casei MG4584 (accession number: KCTC14423BP) and L. reuteri MG5346 (accession number: KCTC14507BP).

In addition, the strain may further include Bi. bifidum MG731 (accession number: KCTC13452BP).

The heat-killing process may be heat treatment of a lactic acid bacteria culture, preferably at a temperature of 70° C. to 130° C. for 10 minutes to 60 minutes, and more preferably at a temperature of 90° C. to 110° C. for 20 minutes to 40 minutes.

The preparing method of the composition may further include lyophilizing and powdering the heat-killed strains. The killed strains may also be coated with cryoprotectants such as trehalose, maltodextrin, starch and skim milk powder before lyophilization.

The composition is preferably prepared by mixing L. casei MG4584 killed strain and L. reuteri MG5346 killed strain in the cell number ratio of 1:1 to 2, or the L. casei MG4584 killed strain, the L. reuteri MG5346 killed strain, and the Bi. bifidum MG731 killed strain in the cell number ratio of 1:1 to 2:1 to 2 at concentrations of 1×10⁸ to 1×10¹⁰ cells/ml, respectively, or finally adjusting the composition including the mixed strain to have a killed strain concentration of 1×10⁸ to 1×10¹⁰ cells/ml using an appropriate dilute.

Hereinafter, the present disclosure will be described in more detail with reference to Example embodiments. These Example embodiments are only illustrative for the present disclosure, and it will not be understood that the scope of the present disclosure is limited by these Example embodiments.

Preparation of Materials

1. Preparation of Killed Strains

Lactic acid strains used in an experiment were provided by MEDIOGEN Co., Ltd. (Jecheon, Korea). The strains were inoculated into an MRS medium, incubated at 37° C. for 18 hours, centrifuged (5,000×g, 4° C., 5 minutes), and the strains were washed three times with distilled water. The washed strains were added with sterile distilled water to prepare a suspension, and then heat-treated at 100° C. for 30 minutes. Then, through rapid lyophilization at −80° C. and grinding, killed strain powder was obtained and stored in a refrigerator until use in the experiment.

The lactic acid bacteria used for preparing the killed strain powder were Bi. bifidum MG731, L. bulgaricus MG515, L. casei MG311, L. casei MG4584, L. plantarum MG4215, L. reuteri MG5346 and L. rhamnosus MG316.

2. Cell Culture

Human colorectal carcinoma RKO cells were obtained from American Type Culture Collection (ATCC; Manassas, Virginia, USA) and incubated in a DMEM medium (Corning Inc, New York, USA) containing 10% fetal bovine serum (FBS, Young-In Frontier, Seoul, Korea) and 1% penicillin/streptomycin (Gibco, Grand Island, USA) under conditions of 37° C. and 5% CO₂.

### Example Embodiment 1: Apoptotic Effect of Killed Strains on Colorectal Cancer Cells

Apoptosis assay was performed using an Annexin V-FITC Apoptosis Detection Kit I (Roche, CA, USA).

The RKO cells were incubated overnight in a 96-well culture plate (1×10⁶ cell/well), and treated with previously prepared 7 types of killed strains at a concentration of 1×10⁹ cells/ml for 24 hours, and then the cells were collected and subjected to Annexin V-FITC and Propidium iodide (PI) staining. After incubation in the dark at room temperature for 15 minutes, the cells were immediately analyzed by flow cytometry (EasyCyte guava, Merck Millipore).

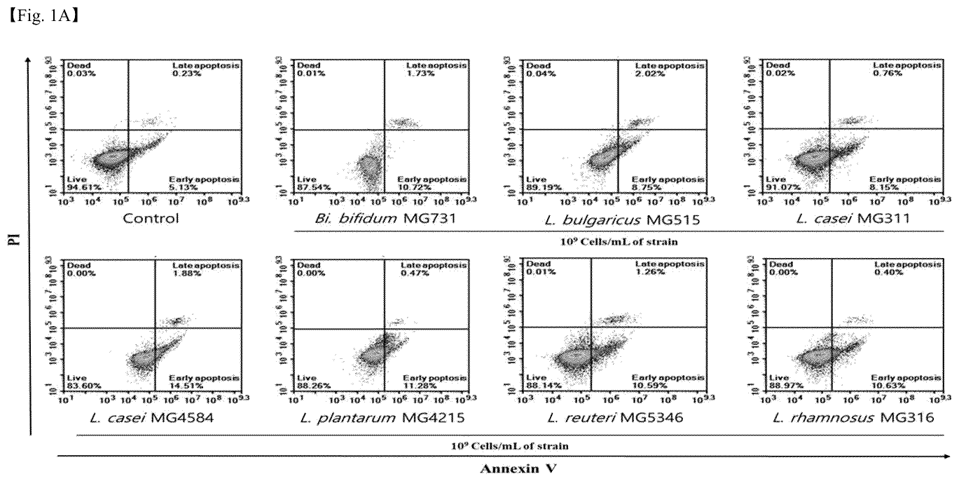

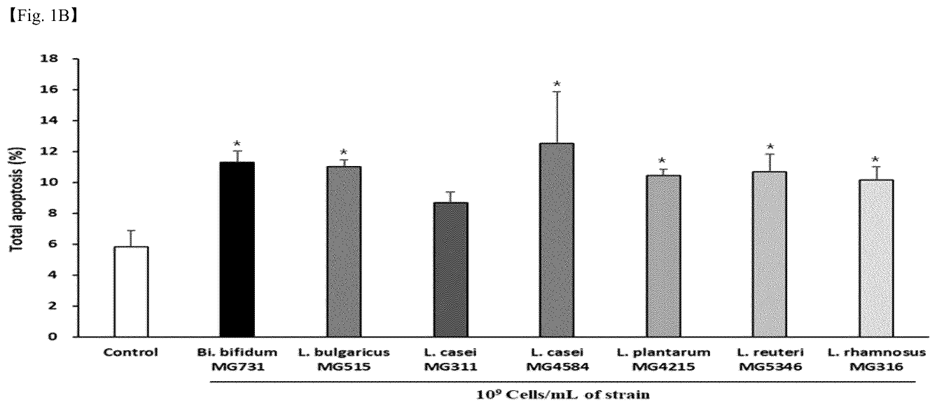

The percentage of the analyzed cells was illustrated in FIGS. 1A-1B. As illustrated in FIG. 1A, the cells were classified into four categories of dead cells stained only with PI (Dead), late apoptosis stained with Annexin V-FITC and PI (Late apoptosis), early apoptosis stained only with Annexin V-FITC (Early apoptosis), and unstained live cells (Live) on quadrants in clockwise from the top left. The sum of early apoptosis (%) and late apoptosis (%) of average data analyzed three times was quantified as total apoptosis (%), which was shown in a graph of FIG. 1B.

Referring to FIG. 1B, it was confirmed that when the killed strain of L. casei MG4584 was treated, apoptosis induction was significantly increased, and the killed strains of other strains such as Bi. bifidum MG731, L. reuteri MG5346, and L. plantarum MG4215 effectively induced apoptosis compared to a control group.

### Example Embodiment 2: Tumor Inhibitory Effect of Mixed Strain on Xenograft Animal Models

Bi. bifidum MG731, L. casei MG4584 and L. reuteri MG5346, which were confirmed to have an apoptotic effect on RKO cells in Example embodiment 1, were selected, and antitumor effects of killed strains and mixed killed strains (2Mix and 3Mix) of each of the lactic acid bacteria were compared and analyzed using a xenograft animal model of RKO cells.

Animal experiments were approved by the Animal Experimentation Committee of Duksung Women's University (permission number: 2021-005-005) and performed in accordance with the guidelines of the Institutional Animal Care and Use Committee (IACUC, Seoul, Korea).

BABL/c rats (female, 5-week-old; Raonbio Co. Ltd., Seoul, Korea) were used as a xenograft animal model. The rats were individually housed in a pathogen-free controlled environment (23 to 27° C. and 45±5% humidity on a 12 hr day/12 hr night cycle) and provided arbitrarily with standard laboratory food and water. Human colorectal cancer RKO cells (1×10⁶ cells/mouse) were subcutaneously injected into the back of the right hind leg of the rat, and then the killed strain powder was dissolved in drinking water and orally administered daily for 3 weeks. The rats were randomly assigned to 6 groups and shown in Table 1 (n=8/group).

TABLE 1

| | | |
|---|---|---|
| 1 | Control | Water |
| 2 | MG731 group | Bi. bifidum MG731 (1 × 10⁹ cells/mouse) |
| 3 | MG5346 group | L. reuteri MG5346 (1 × 10⁹ cells/mouse) |
| 4 | MG4584 group | L. casei MG4584 (1 × 10⁹ cells/mouse) |
| 5 | Two-mixed group | L. reuteri MG5346 (1 × 10⁹ cells/mouse) + |
| | (2Mix) | L. casei MG4584 (1 × 10⁹ cells/mouse) |
| 6 | Three-mixed group | Bi. bifidum MG731 (1 × 10⁹ cells/mouse) + |
| | (3Mix) | L. reuteri MG5346 (1 × 10⁹ cells/mouse) + |
| | | L. casei MG4584 (1 × 10⁹ cells/mouse) |

Tumors were confirmed and measured every two days using standard calipers. The tumor volume was calculated as [tumor length (mm)×tumor width (mm)2]/2. When the tumor volume reached 2000 mm³, the rats were euthanized and the tumors were harvested.

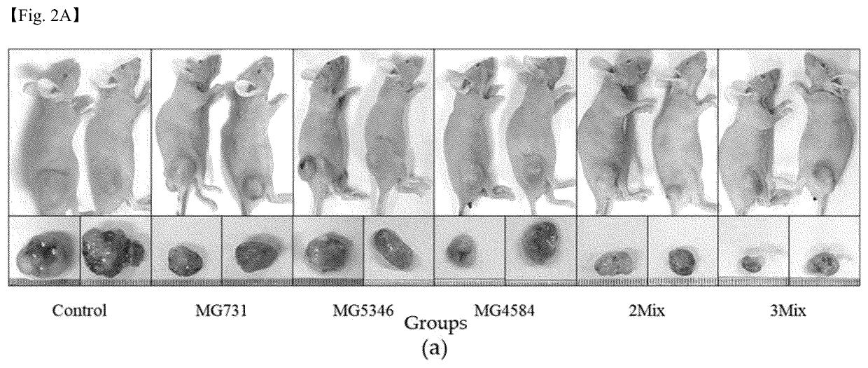

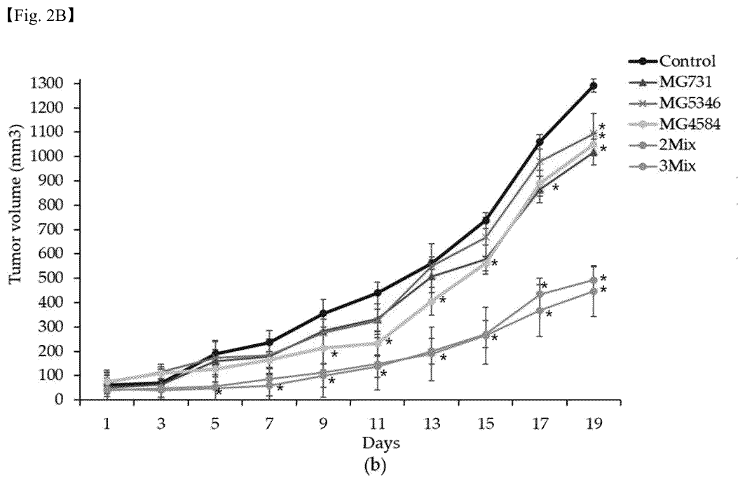

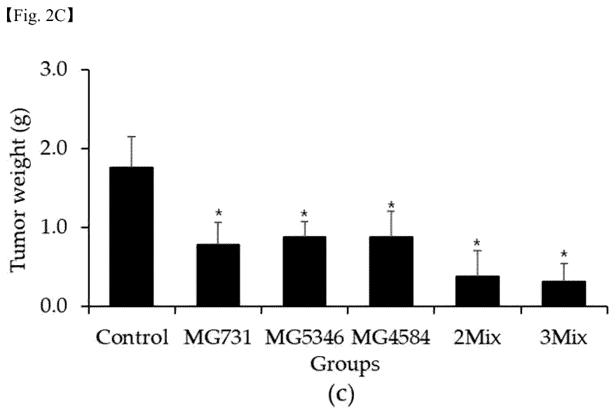

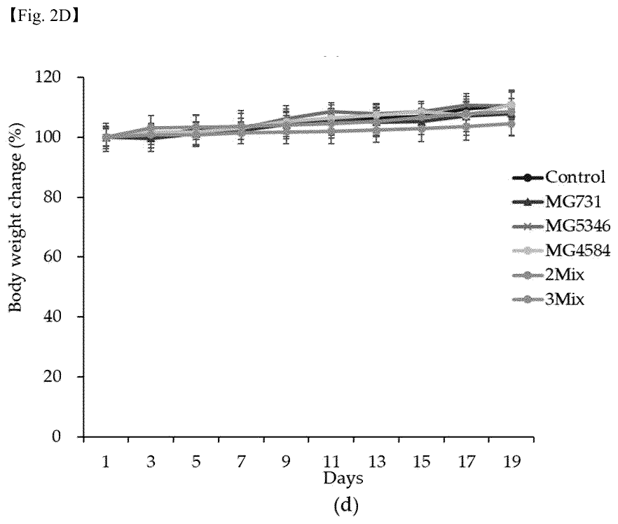

The weights or volumes of tumors collected from 6 rat groups were measured and shown in FIGS. 2A-2D. As shown in FIG. 2A, it may be visually distinguished that the size of the tumor is significantly reduced in the two-mixed group (2Mix) or three-mixed group (3Mix).

Specifically, referring to FIG. 2B, the groups supplied with the dissolved killed strain powder exhibited tumor growth inhibitory activity compared to the control group supplied with only drinking water after treatment with RKO cells from the 5th day of the administration period.

Particularly, it was confirmed that the killed strain two-mixed group (2Mix) and the three-mixed group (3Mix) significantly inhibited the tumor growth from the beginning of administration. At the records on the 19th day for each of the killed strain administered groups, it was confirmed that a single administered group inhibited the tumor growth by approximately 15.18%, and the two-mixed group (2Mix) or three-mixed group (3Mix) significantly decreased the tumor volume by approximately 61.76% or more compared to the control group.

In addition, in changes in tumor weight in FIG. 2C, it was confirmed that all experimental groups showed a positive correlation with the change in tumor volume, and the killed strain powder administered group showed an effect of reducing the tumor weight compared to the control group.

And compared to the single administered group, the mixed killed strain administered group of the two-mixed group (2Mix) or three-mixed group (3Mix) significantly reduced the tumor weight.

As these results, the synergistic effect on colorectal cancer tumor suppression was confirmed in the two-mixed group (2Mix) of L. casei MG4584 and L. reuteri MG5346, or the three-mixed group (3Mix) of Bi. bifidum MG731, L. casei MG4584 and L. reuteri MG5346, and it was confirmed that the combination of the mixed killed strains exhibited excellent preventive or therapeutic effects of colorectal cancer.

Meanwhile, it was confirmed that there was no general toxicity by oral administration of killed strains as a record of a steady increase in body weight in all groups during the experimental period (FIG. 2D).

### Example Embodiment 3: Apoptosis-Related Signal Activity Effect of Mixed Killed Strains in Tumors

In Example embodiment 2, the expression levels of apoptosis-related proteins were analyzed in the two-mixed group (2Mix) or the three-mixed group (3Mix), which showed excellent tumor growth inhibitory activity. Specifically, a caspase cascade pathway including caspase-9, -3, -7 and PARP, which were known to be involved in tumor apoptosis, was identified.

Tumor tissue specimens of the control group and the mixed killed strain administered groups (2Mix and 3Mix) were dissolved in an extraction buffer (Intron Biotechnology, Seoul, Korea) to extract proteins, and the extracted proteins were quantified using Coomassie (Bradford) Protein Assay (genDEPOT, Katy, Texas, USA). The proteins were separated by electrophoresis and transferred to a nitrocellulose membrane (0.45 μM pore size, Merck Millipore, Burlington, Massachusetts, USA) and then blotted the membrane with primary and secondary antibodies.

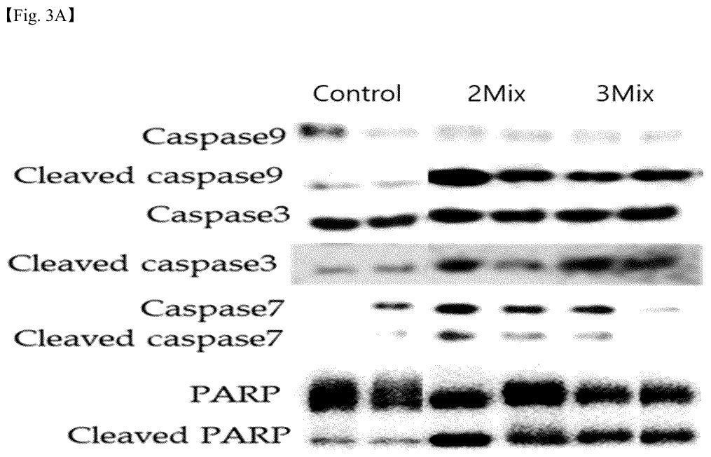

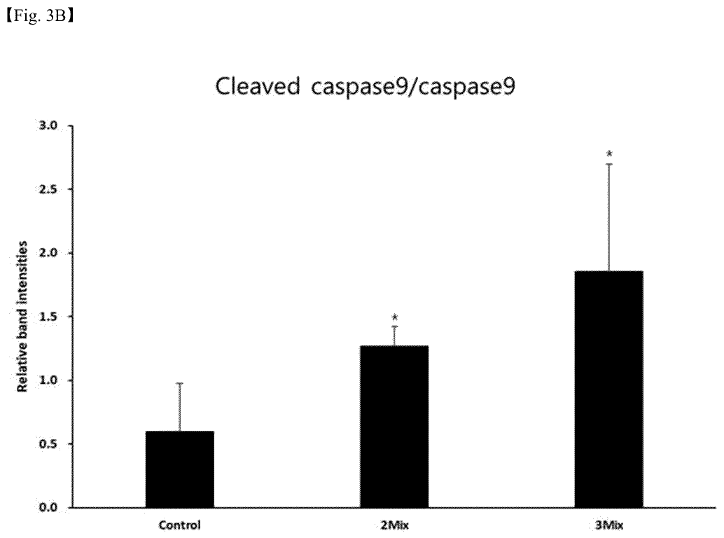

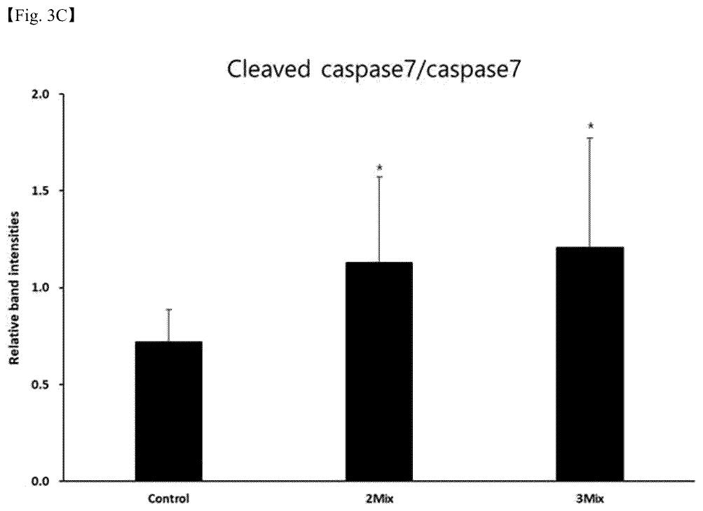

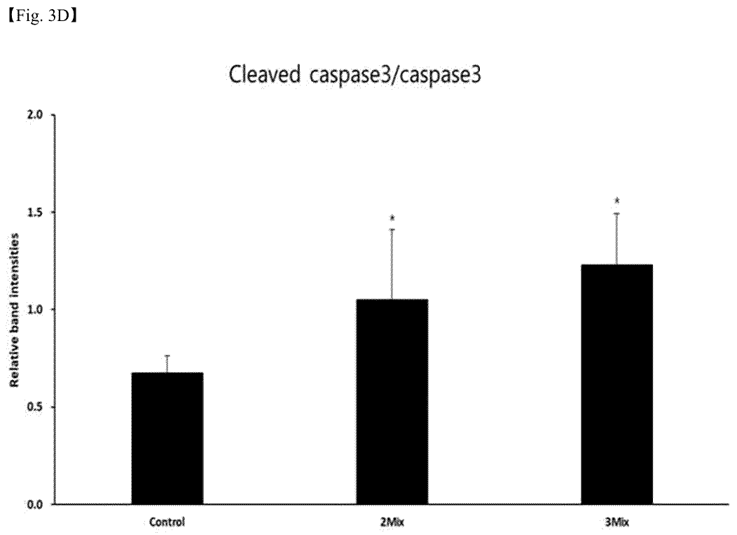

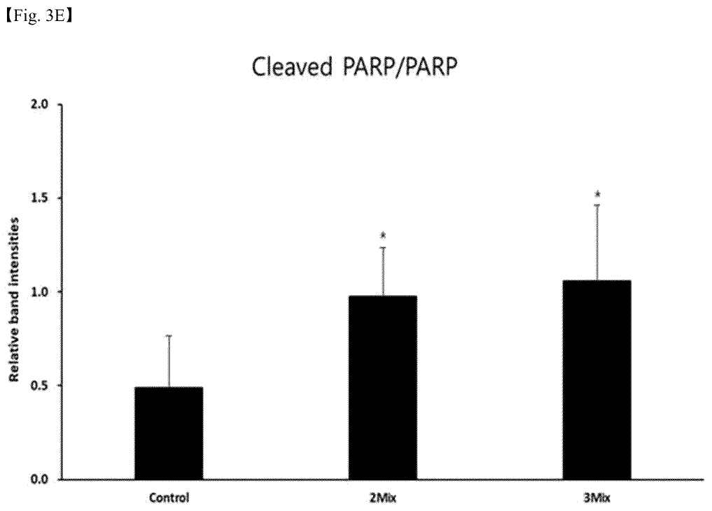

The data of the proteins visualized by chemiluminescence detection (ECL System, Bio-Rad, Hercules, California, USA) and quantified using the Image J program (National Institute of Health, Bethesda, Maryland, USA) were shown in FIGS. 3A-3E.

As shown in FIGS. 3A to 3E, it was confirmed that in RKO cell-derived tumor tissues, both the two-mixed group (2Mix) and the three-mixed group (3Mix) significantly increased the expression levels of cleaved caspase-9, caspase-3, caspase-7 and PARP as compared to the control group supplied with only drinking water.

### Example Embodiment 4: Immunohistochemical Analysis of Tumor Tissue

A caspase cascade of the mixed killed strain administered groups (2Mix and 3Mix) was additionally confirmed by immunohistochemical (IHC) staining of the tumor tissues of RKO cell xenograft rats.

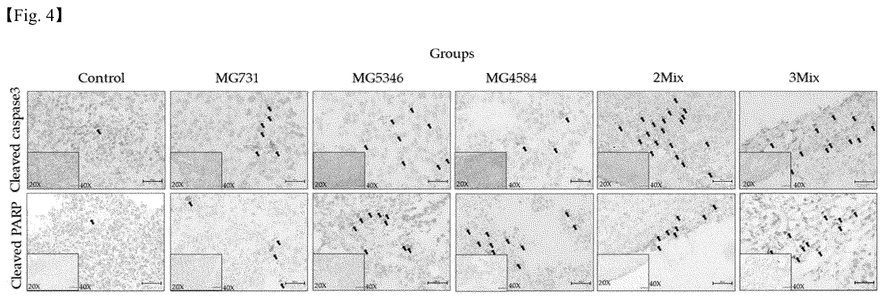

Tumor tissues in 6 groups collected in Example embodiment 2 were frozen with a frozen section compound (Leica, Hessen, Germany) and stored at −80° C. Tumor tissue blocks were cut into 4 μm-thick sections and then the sections were incubated overnight at 4° C. with primary antibodies. The sections were diluted at 1:500 and 1:50 in TBST, respectively, and slices were incubated with a secondary antibody for 1 hour at room temperature. Thereafter, the slices were stained with DAB (Vector Laboratories, Burlingame, California, USA) and visualized under a microscope (×200) as shown in FIG. 4.

Referring to FIG. 4, it may be seen that the expression levels of cleaved caspase-3 and cleaved PARP were significantly increased in the two-mixed group (2Mix) and the three-mixed group (3Mix) compared to the single killed strain administered group.

The results confirmed in Example embodiments 3 and 4 mean that the tumor growth inhibitory activity of the mixed killed strain confirmed in Example embodiment 2 is the activity by apoptosis induction of the mixed killed strain other than the necrosis of the tumor, and it was confirmed that the combination of the killed strains of the present disclosure exhibited excellent apoptotic effect and antitumor activity on colorectal cancer.

[Accession Number]

Depositary Authority Name: Korean Collection for Type Cultures (KCTC)

Accession number: KCTC13452BP

Accession Date: 20180104

Depositary Authority Name: Korean Collection for Type Cultures (KCTC)

Accession number: KCTC14423BP

Accession Date: 20210105

Depositary Authority Name: Korean Collection for Type Cultures (KCTC)

Accession number: KCTC14507BP

Accession Date: 20210322

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method for alleviating or treating colorectal cancer, comprising:
administering a composition, to a subject, including a mixture of a killed strain of L. casei MG4584 (accession number: KCTC14423BP) and a killed strain of L. reuteri MG5346 (accession number: KCTC14507BP) or a culture thereof.

2. The method for alleviating or treating colorectal cancer of claim 1, wherein the composition further includes a killed strain of Bi. bifidum MG731 (accession number: KCTC13452BP) or a culture thereof.

3. The method for alleviating or treating colorectal cancer of claim 2, wherein the killed strain of L. casei MG4584, L. reuteri MG5346 or Bi. bifidum MG731 is heat-killed.

4. The method for alleviating or treating colorectal cancer of claim 3, wherein the heat-killing is heat treatment at a temperature of 70° C. to 130° C. for 10 minutes to 60 minutes.

5. The method for alleviating or treating colorectal cancer of claim 1, wherein the killed strain of L. casei MG4584 and the killed strain of L. reuteri MG5346 are included in a cell number ratio of 1:1 to 2.

6. The method for alleviating or treating colorectal cancer of claim 2, wherein the killed strain of L. casei MG4584, the killed strain of L. reuteri MG5346, and the killed strain of Bi. bifidum MG731 are included in a cell number ratio of 1:1 to 2:1 to 2.

7. The method for alleviating or treating colorectal cancer of claim 1, wherein the composition has tumor growth inhibitory activity or apoptosis inducing activity of cancer cells.

8. The method for alleviating or treating colorectal cancer of claim 1, wherein the composition is a pharmaceutical composition, a health functional food composition or a feed additive composition.

9. The method for alleviating or treating colorectal cancer of claim 8, wherein the pharmaceutical composition further comprises a pharmaceutically acceptable carrier, excipient or diluent.

10. A method for preparing a composition for alleviating or treating colorectal cancer, comprising:
mixing heat-killed strains,
wherein the strains are L. casei MG4584 (accession number: KCTC14423BP) and L. reuteri MG5346 (accession number: KCTC14507BP).

11. The method for preparing a composition for alleviating or treating colorectal cancer of claim 10, wherein the strain further includes Bi. bifidum MG731 (accession number: KCTC13452BP).

12. The method for preparing a composition for alleviating or treating colorectal cancer of claim 10, further comprising:
lyophilizing and powdering the heat-killed strains.

13. The method for preparing a composition for alleviating or treating colorectal cancer of claim 10, wherein the killed strain of L. casei MG4584 and the killed strain of L. reuteri MG5346 are included in a cell number ratio of 1:1 to 2.

14. The method for preparing a composition for alleviating or treating colorectal cancer of claim 11, wherein the killed strain of L. casei MG4584, the killed strain of L. reuteri MG5346, and the killed strain of Bi. bifidum MG731 are included in a cell number ratio of 1:1 to 2:1 to 2.
